(19) United States
(12) Patent Application Publication
(10) Pub. No.: US 2019/0224478 A1
(43) Pub. Date: Jul. 25, 2019

(54) HIGH-FREQUENCY HANDPIECE FOR SKIN TREATMENT

(71) Applicant: PANACE CO., LTD., Seongnam-si, Gyeonggi-do (KR)

(72) Inventor: Sung Tae YOON, Seoul (KR)

(73) Assignee: PANACE CO., LTD., Seongnam-si, Gyeonggi-do (KR)

(21) Appl. No.: 16/314,890

(22) PCT Filed: Jul. 4, 2017
(86) PCT No.: PCT/KR2017/007102

§ 371 Date: Jan. 3, 2019

(30) Foreign Application Priority Data
Jul. 4, 2016 (KR) 10-2016-0084225

(51) Int. Cl. A61N 1/32; A61N 1/06; A61N 1/05

(52) U.S. Cl. CPC A61N 1/328; A61N 1/0504; A61N 1/0502; A61N 1/06

(57) ABSTRACT

A high frequency handpiece for skin treatment is provided, which includes: a handle of which one end is connected to a high frequency generator and the other end has an opening formed thereon, in which the handle has a cylindrical shape to allow grip by hand; and a needle cartridge fitted to one side of the handle and including a needle formed thereon to output a high frequency to skin when supplied with power, in which the needle cartridge includes a holder having both open sides and a passage formed therein; a needle assembly coupled to one side of the holder and having the needle formed therein to be inserted into the skin; a connector coupled to an opening on the other side of the holder, connected to one side of the needle assembly, and having a fitting part formed at the other side; and a power plug removably fitted into the fitting part of the connector and is coupled to the handle to supply power. Accordingly, because the cartridge having needles integrally formed thereon can be separated from the handle, replacement with a new cartridge can be facilitated, and the needles can be replaced in a more hygienic manner.

### CROSS-REFERENCE TO RELATED APPLICATIONS

The present disclosure claims priority to Korean Patent Application No. 10-2016-0084225 filed on 4 Jul. 2016 in the Republic of Korea, the disclosures of which are incorporated herein by reference.

### BACKGROUND

### 1. Field of the Invention

The present disclosure relates to a high frequency handpiece for skin treatment, and more particularly, to a high frequency handpiece for skin treatment, which directly transmits a high frequency current to the dermal layer between the epidermal layer and the subcutaneous fat layer of the skin to activate the cell tissue, thus enabling elastic skin to be maintained and skin aging to be minimized.

### 2. Description of the Related Art

Skin covers our entire body and is formed of three layers including the epidermal layer, the dermal layer, and the subcutaneous fat layer. The epidermal layer is the outermost layer of the skin, which is divided into several layers including cornified layer, clear layer, granular layer, spinous layer, and basal layer according to their locations and functions, and provides function of protection, defense, secretion, etc. The dermal layer is located below the epidermal layer and adjacent to the base layer, constitutes the majority of the skin, and is divided into: papillary dermis composed of water, proteins, saccharides, mucopolysaccharides, minerals, and inorganic salts in gel-like state, which has capillary vessels associated with blood circulation and lymphatic vessels carrying lymph; and reticular dermis composed of collagen which is collagenous fiber associated with the wrinkles of the skin, elastin which is resilient fiber that provides elasticity to the skin, and ground substrate (reservoir of water). Finally, the subcutaneous fat layer is located between the dermis, muscles and bones, contains a large amount of fat, and forms the lowest layer of the skin. The subcutaneous fat layer spreads evenly throughout the human body, providing elasticity-maintaining and buffering action of absorbing external pressure and impact, thereby not only protecting the inside of the body against damages, but also preventing the loss of body heat and maintaining body temperature.

In order to prevent the aging of the skin described above, besides the traditional method of massaging skin with a cosmetic material containing nutrients, a method of injecting a serum into skin using a syringe or the like is widely used. However, this skin treatment method has disadvantage that it is not only costly, but also can have many side effects depending on the physical constitution of the treated person, in which case it may rather adversely affect the skin.

Additionally, a method of indirectly delivering high frequency or the like from the epidermal layer to the dermal layer of the skin is also used, but this method has a shortcoming in that high frequency is indirectly transmitted to the dermal layer through the epidermal layer, thus providing less satisfactory effect compared to the massage-type skin treatment method, and also causing inconvenience and high cost burden by requiring treatment to be continued periodically.

High frequency skin treatment method, which transmits high frequency currents to activate cell tissue directly to the dermal layer of the skin through the use of needle, thus maintaining skin elasticity and minimizing aging of skin, is receiving increasing attention. An example can be found in Korean Patent Laid-Open No. 10-2015-0007938, entitled “Skin treatment apparatus”. The related art disclosed in Korean Patent Laid-Open No. 10-2015-0007938 includes a treatment unit which is inserted into a patient's skin to stimulate the skin, a holder to which the treatment unit is removably attached and which transmits a signal to the treatment unit, and a handpiece to which a holder part is mounted and which supplies a signal to the holder. The treatment unit includes a treatment needle inserted into the skin; a support plate connected to the treatment needle and restricted from moving by the skin; and an insertion plate connected to the support plate and mounted on the holder. To prevent risks such as the transmission of germs, used treatment unit is discarded and replaced with a new one. The replacement of the treatment unit with a new treatment unit after every use involves a process of gripping and separating a small-sized treatment unit with tweezers, and then replacing with a new one, which is a very inconvenient and difficult task, and there is also a high risk of secondary infection when the treatment unit is lost.

### SUMMARY

The present disclosure is designed to solve the problems described above, and it is an object of the present disclosure to provide a high frequency handpiece for skin treatment, in which a cartridge with a needle for insertion into the skin is easily separated from and coupled to a handle, thus solving difficulty related with the replacement of the needle, and enabling more hygienic replacement, and also preventing a risk of accident due to misplaced needle.

The object of the present disclosure described above can be achieved by a high frequency handpiece for skin treatment, which may include: a handle of which one end is connected to a high frequency generator and the other end has an opening formed thereon, in which the handle has a cylindrical shape to allow grip by hand; and a needle cartridge removably coupled to one side of the handle and including a needle formed thereon to output a high frequency to skin when supplied with power, in which the needle cartridge includes a holder having both open sides and a passage formed therein; a needle assembly coupled to one side of the holder and having the needle formed therein to be inserted into the skin; a connector coupled to an opening on the other side of the holder, connected to one side of the needle assembly, and having a fitting part formed at the other side; and a power plug removably fitted into the fitting part of the connector and is coupled to the handle to supply power.

The holder may include a coupling part including a needle insertion hole formed at one side for receiving the needle assembly to be inserted therein, and a through hole formed at the other side for receiving a connector to be inserted therein, and the coupling part may include a fitting slot in a ‘ [symbol] ’ shape on an outer circumferential surface thereof, for coupling with a fastening portion of the handle.

The fastening part of the handle includes a fastening hole formed at one end, into which the fastening part is inserted, and a stopper formed on an inner circumferential surface of the fastening hole to be inserted into the fitting slot.

The connector has a shape of a rod formed of a conductive metal material, and includes the fitting part formed at one end thereof with a plurality of cuts arranged in a circumferential direction, and a mounting surface formed on the other side along an outer circumferential surface for close contact with the needle.

According to the present disclosure, because cartridge having needle integrally formed therein can be easily separated from the handle to be replaced with a new one, difficulty arising from the replacement of the needle can be solved, and hygienic replacement of the needle can be provided, which prevents the risk of secondary infection, and also prevents loss of a fine needle and undesirable accident that may be caused by the misplaced needle.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a perspective view showing a high frequency handpiece for skin treatment according to an embodiment of the present disclosure;

FIG. 2 is an exploded perspective view showing a high frequency handpiece for skin treatment according to an embodiment of the present disclosure;

FIG. 3 is a partially enlarged perspective view of FIG. 2; and

FIGS. 4 and 5 are partial cross-sectional views showing an interior of a high frequency handpiece for skin treatment according to an embodiment of the present disclosure.

### DETAILED DESCRIPTION

According to an embodiment of the present disclosure, a high frequency handpiece for skin treatment may include: a handle of which one end is connected to a high frequency generator and the other end has an opening formed thereon, in which the handle has a cylindrical shape to allow grip by hand; and a needle cartridge fitted to one side of the handle and including a needle formed thereon to output a high frequency to skin when supplied with power, in which the needle cartridge includes a holder having both open sides and a passage formed therein; a needle assembly coupled to one side of the holder and having the needle formed therein to be inserted into the skin; a connector coupled to an opening on the other side of the holder, connected to one side of the needle assembly, and having a fitting part formed at the other side; and a power plug removably fitted into the fitting part of the connector and is coupled to the handle to supply power.

Hereinafter, preferred embodiments of the present disclosure will be described in detail with reference to the accompanying drawings. It should be noted that the same elements are denoted by the same reference numerals as possible in the accompanying drawings. Further, the detailed description of well-known functions and configurations that may obscure the gist of the present disclosure will be omitted.

In addition, it should be understood that the terms used in the description and the appended claims which will be described below should not be construed as limited to general and dictionary meanings, but interpreted based on the meanings and concepts corresponding to technical aspects of the present disclosure on the basis of the principle that the inventor is allowed to define terms appropriately for the best explanation.

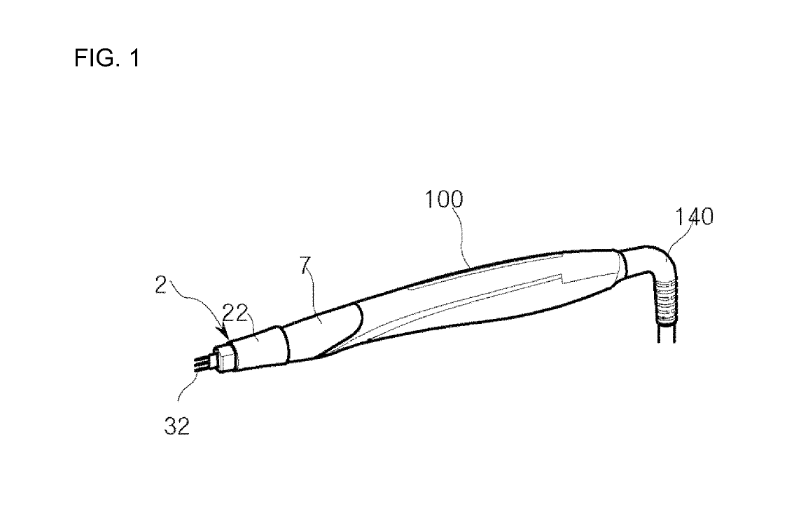

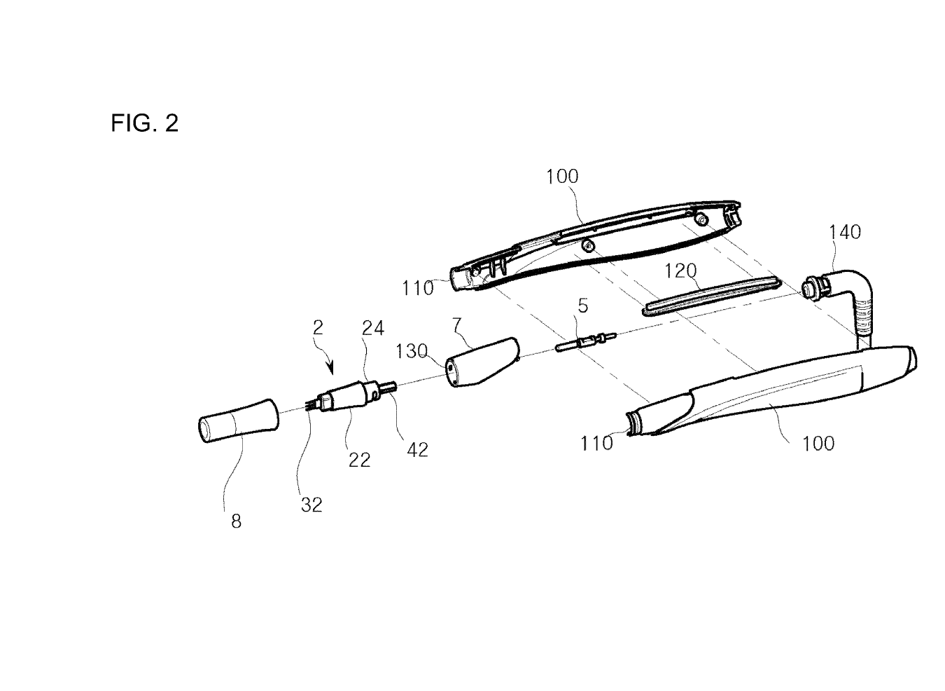

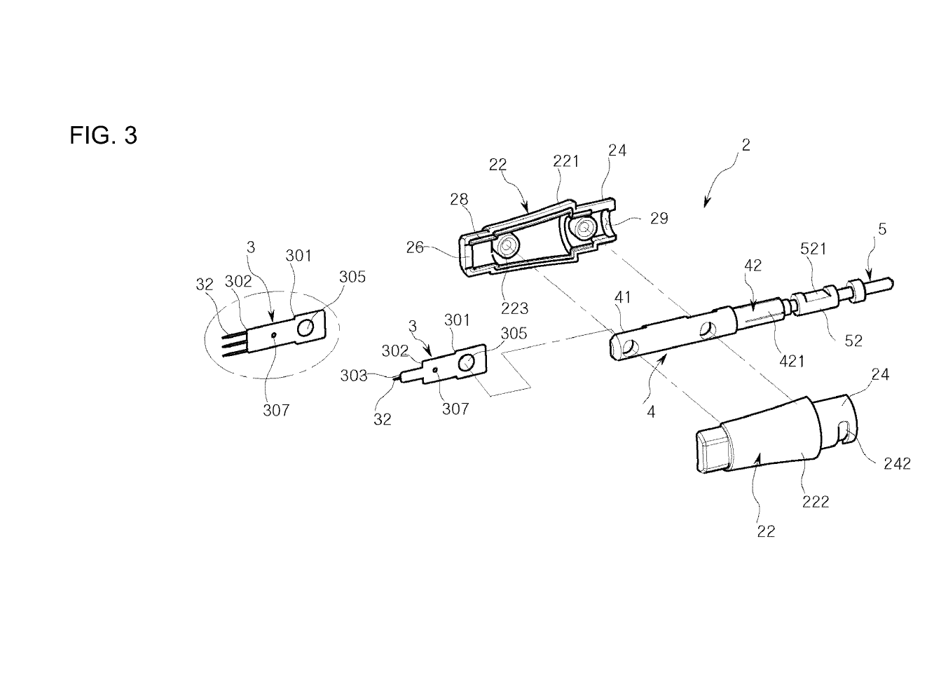

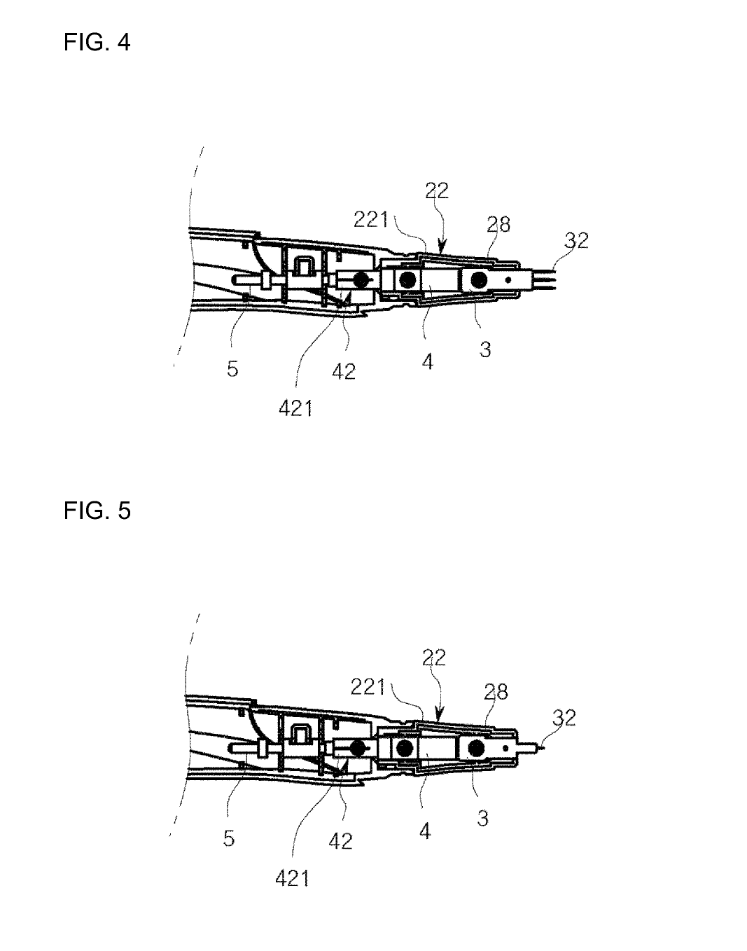

In the accompanying drawings, FIG. 1 is a perspective view showing a high frequency handpiece for skin treatment according to an embodiment of the present disclosure, FIG. 2 is an exploded perspective view showing a high frequency handpiece for skin treatment according to an embodiment of the present disclosure, FIG. 3 is a partially enlarged perspective view of FIG. 2, and FIGS. 4 and 5 are partial cross-sectional views showing an interior of a high frequency handpiece for skin treatment according to an embodiment of the present disclosure.

The high frequency handpiece for skin treatment according to an embodiment of the present disclosure includes a handle 100 having a cylindrical shape to allow grip by hand, of which one end is connected to a high frequency generator (not illustrated), and the other end has an opening formed therein; and a needle cartridge 2 fitted into one side of the handle 100 and including a needle 32 for outputting high frequency to the skin when supplied with power.

The handle 100 has a predetermined length and has a cylindrical shape, while having a streamlined shape to provide good grip feeling and to prevent slippage. In addition, the handle 100 may be divided into two parts, and house therein parts such as a conductor 120 and a power plug 140 for supplying power to inside.

A cap 7 is coupled to a front end of the handle 100 for fitting with the coupling part 24 of the needle cartridge 2. A stopper 130 is formed on an inner circumferential surface of the cap 7 to be inserted into a fitting slot 242.

The needle cartridge 2 is removable, that is, the needle cartridge 3 is attachable to and detachable from the handle 100 and thus can be discarded and replaced with a new one after every use.

The needle cartridge 2 includes: a holder 22 having both open sides and a passage formed therein; a needle assembly 3 coupled to one side of the holder 22 and having the needle 32 therein to be inserted into the skin; a connector 4 coupled to the other opening of the holder 22 and connected to one side of the needle assembly 3, and having a fitting part 42 formed on the other side; and a power plug 140 removably fitted into the fitting part 42 of the connector 4 and coupled to the handle 100 to supply power.

The holder 22 includes a needle insertion hole 26 formed at one side to receive the needle assembly 3 to be inserted thereinto, and the coupling part 24 formed at the other side, having a through hole 29 to receive the connector 4 to be inserted thereinto.

The holder 22 is formed as a cylindrical body that is formed by coupling two sub-bodies, i.e., first and second bodies 221 and 222 to face each other, and has a tapered shape such that the diameter increases from a front end where the needle insertion hole 26 is formed, toward a rear end where the coupling part 24 is formed, thus allowing easy grip with the thumb and index finger.

The coupling part 24 is formed in a cylindrical shape at the rear end of the holder 22, and is formed in a smaller diameter than the rear end of the holder 22, and has symmetrical fitting slots 242 recessed into a concave shape that is a′ L′ shape on the outer circumferential surface of the coupling part 314 to receive the coupling part 110 of the handle 100 to be fitted therein.

The stopper 130 is formed on an inner circumferential surface of the cap 7 to be inserted into the fitting slots 242.

Spaces are formed inside the first and second bodies 221 and 222, and guide steps 28 are formed on a cutting plane of the first and second bodies 311 and 312, along edges of the spaces.

The guide steps 28 are configured for a close contact with an outer periphery of the needle assembly 3 to be described below. Because the needle assembly 3 can be brought into close contact with the guide steps 28, the needle assembly 3 can be stably secured.

The needle assembly 3 is a thin metal plate having conductivity, and includes a through hole 29 formed at one side to be coupled to the connector 4, and the sharp-pointed needle 32 formed at the other end.

The needle assembly 3 is formed such that width thereof decreases by one or two levels starting from a portion opposite the needle 32, into a first step 301, a second step 302 and a third step 303.

The first to third steps 301 to 303 are shaped to fit the guide steps 28 of the holder 22.

The needle assembly 3 includes a through hole 305 formed at one side, through which a coupling shaft 223 that is passed through holder 22 and the connector 4 is passed, and a fixing hole 307 formed on the other side, into which a pin formed in the holder 22 is fitted.

Therefore, when the needle assembly 3 is coupled to the holder 22 and then coupled to the connector 4, the needle assembly 3 can be firmly secured so as not to be moved.

The needle 32 is sharply-pointed to be inserted into the skin, and is formed of a conductive material to receive a high frequency signal from the outside and transmit the same to the skin.

One, or two or more, that is, a plurality of needles 32 may be provided. FIG. 4 shows an example where there are three needles 32, and FIG. 5 shows an example where there is only one needle 32.

The connector 4 is a circular rod with conductivity, which has a mounting surface 41 formed at one side by vertically cutting the outer circumferential surface, and a plurality of cuts 421 arranged in a circumferential direction on the other side having hollow cylindrical shape, that is, on the fitting part 42.

The needle assembly 3 is brought into close contact with the mounting surface 41 of the connector 4.

The connector 340 and the needle assembly 3 may be integrated as a screw is coupled through the mounting surface 41, with a portion of the screw being coupled to the needle assembly 300.

The power plug 140 is a circular rod with conductivity, of which one end is coupled to the fitting part 42 of the connector 4 by interference-fitting and the other end is connected to the conductor 120 by fitting.

The power plug 140 has an extended portion 52 in the middle portion where the outer diameter is increased, and a flat portion that forms a groove portion 521. The groove portion 521 is inserted into the conductor 120 to serve as a latching jaw, thus preventing the power plug 140 from falling off accidentally.

Hereinafter, the operation of the present disclosure with the configuration described above will be described.

The connector 4 is inserted into one sides of the first and second bodies 221 and 222 constituting the holder 22, and then the needle assembly 3 is inserted into the holder 22 on the other side. Then, the first and second bodies 221 and 222 are coupled to each other by screw-fastening, according to which both the needle assembly 3 and the connector 4 are also screw-fastened and integrated. As a result, the needle cartridge 2 is assembled.

The assembled needle cartridge 2 is fitted into the fastening part 110 of the handle 100. At this time, the cap 7 is engaged with a leading end of the handle 100 to improve the fixing force.

Meanwhile, since the needle 32 exposed to the outside can be dangerous, a lid 8 is coupled to one side of the holder 22 to protect the needle 32 in order to prevent an accident and damage to the needle 32.

When it is necessary to use the needle, the user removes the lid 8 and holds the handle 100 and allows the needle 32 to be inserted into the skin, and then applies a high frequency to perform the treatment.

After completing the treatment, the user rotates the holder 22 to cause the stopper 130 to be separated from the fitting slot 242, and then pulls the holder 22 outward to separate the needle cartridge 2 from the handle 100.

Therefore, the used needle cartridge 2 can be discarded and the new needle cartridge 2 can be coupled to the handle 100 to be used, thereby ensuring hygienic process and safety.

While the embodiments of the present disclosure have been described with reference to the accompanying drawings, those with ordinary knowledge in the technical field of the present disclosure will be able to understand that the present disclosure can be embodied into difference and more detailed modes, without departing from the technical concept or without modifying essential characteristics thereof. Accordingly, it will be understood that the exemplary embodiments described above are only illustrative, and should not be construed as limiting.

### INDUSTRIAL APPLICABILITY

The present disclosure relates to a high frequency handpiece for skin treatment and can be used as a medical device for skin treatment.

What is claimed is:

1. A high frequency handpiece skin treatment, comprising:
a handle, of which one end is connected to the high frequency generator and the other end has an opening formed therein;
a needle cartridge removably coupled to one side of the handle and having a needle formed therein to be inserted into skin and transmit a high frequency to the skin,
wherein the needle cartridge comprises:
a holder having both open sides and a passage formed therein;
a needle assembly coupled to one side of the holder and having the needle formed therein;
a connector coupled to an opening on the other side of the holder, connected to one side of the needle assembly, and having a fitting part formed at the other side; and
a power plug removably fitted into the fitting part of the connector and is coupled to the handle to supply power.

2. The high frequency handpiece skin treatment according to claim 1, wherein the holder comprises a coupling part including a needle insertion hole formed at one side for receiving the needle assembly to be inserted therein, and a through hole formed at the other side for receiving a connector to be inserted therein, and
the coupling part includes a fitting slot in a ‘ [symbol] ’ shape on an outer circumferential surface thereof, for coupling with a fastening portion of the handle.

3. The high frequency handpiece skin treatment according to claim 1, wherein the handle comprises
a cap coupled to one side thereof to receive a coupling part of the needle cartridge to be fitted thereinto, and
a stopper formed on an inner circumferential surface of the cap to be inserted into a fitting slot.

4. The high frequency handpiece skin treatment according to claim 1, wherein the connector has a shape of a rod formed of a conductive metal material, and includes the fitting part formed at one end thereof with a plurality of cuts arranged in a circumferential direction, and
a mounting surface formed on the other side along an outer circumferential surface for close contact with the needle.

5. The high frequency handpiece skin treatment according to claim 1, wherein the needle assembly is a thin conductive metal plate and includes through holes formed at one side that is coupled to the connector, and one, or a plurality of sharp-pointed needles at the other end.
